(19) United States
(12) Patent Application Publication
(10) Pub. No.: US 2022/0265902 A1
(43) Pub. Date: Aug. 25, 2022

(54) HYDROGEL WITH ANTICANCER EFFICACY AND METHOD FOR PREPARING THE SAME

(71) Applicant: KOREA ATOMIC ENERGY RESEARCH INSTITUTE, Daejeon (KR)

(72) Inventors: Sang Hyun Park, Seoul (KR); Muhammad Asim Raza, Jeongeup-si (KR)

(21) Appl. No.: 17/680,486

(22) Filed: Feb. 25, 2022

(30) Foreign Application Priority Data
Feb. 25, 2021 (KR) 10-2021-0025288

(51) Int. Cl. A61L 27/52; C08L 5/08; A61L 27/20; A61L 27/54; A61L 27/58

(52) U.S. Cl. CPC A61L 27/52; C08L 5/08; A61L 27/20; A61L 27/54; A61L 27/58; C08L 2312/06; A61L 2300/416; A61L 2300/406

(57) ABSTRACT

The present disclosure provides a hydrogel that has excellent drug delivery ability, is pH-dependent, is biocompatible, and has its own anticancer efficacy as well as biodegradability, and a method for preparing the same. Specifically, the present disclosure provides a hydrogel including carboxymethyl-chitosan (CM-CS) and a hydrophilic synthetic polymer wherein the hydrogel is crosslinked by electron beam irradiation, and a method for preparing the same.

### CROSS-REFERENCE TO RELATED APPLICATION(S)

This application claims benefit of priority to Korean Patent Application No. 10-2021-0025288 filed on Feb. 25, 2021 in the Korean Intellectual Property Office, the disclosure of which is incorporated herein by reference in its entirety.

### BACKGROUND

### 1. Field

The present disclosure relates to a hydrogel having anticancer efficacy and excellent drug delivery ability and a method for preparing the same.

### 2. Description of Related Art

It is very important for drugs such as antibiotics and anticancer agents to maintain a specific concentration in the blood or organs in vivo in order to obtain a targeted therapeutic effect. Therefore, techniques to control the amount of drug release are important, and the need for the development of drug carriers having new physical properties suitable for drug delivery is constantly emerging.

A drug delivery system is a dosage form designed to efficiently deliver a required amount of a drug to minimize the side effects of existing drugs and maximize the efficacy and effect of the drugs. In order to efficiently deliver drugs of macromolecules such as proteins and genes with these drug delivery systems, biocompatible and/or biodegradable hydrogels that may be injected into a body are being developed. These hydrogels are formed through chemical and/or physical crosslinking of polymers, and some crosslinking agents are used for chemical bonding.

In addition, the hydrogel is a material that is attracting attention, hydrogel has a high water content and may be applied to use in various fields by controlling chemical and/or physical properties. In particular, by controlling the biocompatibility of the hydrogel, the hydrogel may be used for human bone, cartilage, skin regeneration, drug delivery, wound treatment, etc., so the demand for the hydrogel for tissue regeneration and application to cell therapy is increasing on a daily basis.

For example, a hydrogel with wettability as in Korean Patent No. 1985368 is being studied, and demand for the technological development for a hydrogel with excellent wettability and biocompatibility is continuously increasing.

### SUMMARY

An aspect of the present disclosure may provide a hydrogel that has excellent drug delivery ability, is pH-dependent, is biocompatible, and has anticancer efficacy, as well as biodegradability.

Another aspect of the present disclosure may provide a method for preparing the hydrogel of the present disclosure as described above.

According to an aspect of the present disclosure, there is provided a hydrogel including carboxymethyl-chitosan (CM-CS) and a hydrophilic synthetic polymer and crosslinked by electron beam irradiation.

According to another aspect of the present disclosure, a method for preparing a hydrogel may include: preparing a composition for preparing the hydrogel by mixing carboxymethyl-chitosan (CM-CS) and a hydrophilic synthetic polymer with water; and crosslinking the hydrogel by irradiating the composition for preparing the hydrogel with an electron beam.

### BRIEF DESCRIPTION OF DRAWINGS

The above and other aspects, features, and advantages of the present disclosure will be more clearly understood from the following detailed description taken in conjunction with the accompanying drawings, in which:

FIG. 1 is a diagram illustrating FTIR analysis results of CM-CS, PVP, and a hydrogel according to an exemplary embodiment in the present disclosure;

FIG. 2A is a diagram illustrating swelling in distilled water of the hydrogel according to the exemplary embodiment in the present disclosure, and FIG. 2B is a diagram illustrating a graph related to the relationship between In(F) and In(t);

FIG. 3 is a diagram illustrating measurement results of the swelling according to the pH of the hydrogel according to the exemplary embodiment in the present disclosure;

FIG. 4A is a diagram illustrating an ionic solution comprising NaCl of the hydrogel according to the exemplary embodiment in the present disclosure, and FIG. 4B is a diagram illustrating measurement results of the swelling according to a concentration of CaCl₂) in an ionic solution of the hydrogel according to the exemplary embodiment in the present disclosure;

FIG. 5 is a diagram illustrating cell viability (%) of a RAW 264.7 cell line for the hydrogel according to the exemplary embodiment in the present disclosure;

FIG. 6 is a diagram illustrating cell viability (%) of a cancerous AGS cell line for the hydrogel according to the exemplary embodiment in the present disclosure; and

FIG. 7 is a diagram illustrating measurement results of drug release ability of a hydrogel according to Example 1 of the present disclosure.

### DETAILED DESCRIPTION

Hereinafter, exemplary embodiments in the present disclosure will be described with reference to the accompanying drawings. However, exemplary embodiments in the present disclosure may be modified in several other forms, and the scope of the present disclosure is not limited to exemplary embodiments to be described below.

The present disclosure provides a hydrogel with excellent drug delivery ability, swelling properties, thermal stability, biodegradability, biocompatibility, and anticancer efficacy.

In detail, the present disclosure provides a hydrogel including carboxymethyl-chitosan (CM-CS) and a hydrophilic synthetic polymer and crosslinked by electron beam irradiation.

In the present disclosure, carboxymethyl-chitosan may be N-carboxymethyl chitosan, O-carboxymethyl chitosan, N,O-carboxymethyl chitosan, or any combination thereof. A backbone of the chitosan has 1-4 glycosidic bonds, in which C—H and C—OH bonds of the chitosan are broken by irradiation, and thus, a molecular weight of the chitosan is lowered. However, the present inventors have completed the present disclosure by confirming that carboxymethyl-chitosan crosslinked by irradiation may maintain a crosslinked structure of a hydrogel while expressing anticancer efficacy.

In the present disclosure, as a polymer capable of preparing a hydrogel, a synthetic polymer can be used. In the present disclosure, as a synthetic polymer, a hydrophilic synthetic polymer may be used. For example, the hydrophilic synthetic polymer may be a synthetic polymer including at least one selected from the group consisting of polyvinyl alcohol, polyacrylate, polyvinylpyrrolidone and polyethylene glycol, but is not limited thereto. Preferably, polyvinylpyrrolidone is used.

Meanwhile, the hydrogel of the present disclosure may include, for example, carboxymethyl-chitosan (CM-CS) and a hydrophilic synthetic polymer in a ratio of 1 to 3 parts by weight of the hydrophilic synthetic polymer per 1 part by weight of carboxymethyl-chitosan, and preferably, in a ratio of 1.5 to 2 parts by weight of the hydrophilic synthetic polymer per 1 part by weight of carboxymethyl-chitosan. There may be a problem in that, when the content of the carboxymethyl-chitosan (CM-CS) is less than the above range, anticancer efficacy tends to be lowered, and when the content of the carboxymethyl-chitosan (CM-CS) exceeds the above range, crosslinking may not be sufficiently achieved as the content of the hydrophilic synthetic polymer becomes insufficient, and thus, the hardness and mechanical strength of the hydrogel are lowered.

In addition, the electron beam may be irradiated at a total dose of 5 to 45 kGy, for example, at a total dose of 10 to 45 kGy, and preferably, 15 to 45 kGy. There may be a problem in that, when the total dose of the irradiated radiation is less than 5 kGy, the hydrogel may not be prepared smoothly due to the insufficient crosslinking, and when radiation is irradiated at a dose exceeding 45 kGy, a polymer chain of the hydrogel may be severely crosslinked which will restrict water absorption capacity of the hydrogel.

Meanwhile, during the preparation of the hydrogel of the present disclosure, water is used, but the hydrogel may be a dried hydrogel after being subjected to a drying process, and for example, may be in a form of a dried hydrogel scaffold, or in addition, may be in a form in which the hydrogel is impregnated with a solution such as a drug or a cosmetic composition.

For example, the hydrogel may be loaded with 1 to 75 parts by weight, and preferably, 35 to 75, for example, 40 to 70 parts by weight of the drug per 100 parts by weight of the hydrogel. A lower limit of the loading amount of the drug is not particularly limited, and the hydrogel of the present disclosure may be sufficiently loaded with drug up to 60 to 75 parts by weight per 100 parts by weight of the hydrogel. In this case, the type of the drug is not particularly limited, and the drug may include at least one selected from the group consisting of ampicillin and kanamycin monosulfate monohydrate, for example. Further, when loaded with a drug with anticancer efficacy, the hydrogel of the present disclosure may express a synergistic effect together with its own anticancer efficacy.

The formulation of the drug that may be loaded into the hydrogel of the present disclosure is preferably a fluidized phase, for example, a liquid phase.

As such, the use of the hydrogel of the present disclosure is not particularly limited, and may be for cosmetics, drug delivery, etc., and preferably, may be for drug delivery.

Meanwhile, according to the present disclosure, there is provided a method for preparing the hydrogel of the present disclosure.

In detail, the present disclosure provides a method for preparing a hydrogel including preparing a composition for preparing the hydrogel by mixing carboxymethyl-chitosan (CM-CS) and a hydrophilic synthetic polymer with water; and crosslinking the hydrogel by irradiating the composition for preparation of the hydrogel with an electron beam.

Detailed descriptions related to each component and content in the method for preparing a hydrogel have already been described in the above-described hydrogel, and thus, will be omitted below.

Meanwhile, in the preparing of the composition for preparing the hydrogel by mixing the carboxymethyl-chitosan (CM-CS) and the hydrophilic synthetic polymer with the water, the composition for preparing the hydrogel may be prepared in a single process of mixing all of these components together, or may be sequentially prepared by preparing a solution in which each component is mixed with water and then mixing them. The mixing order and method are not particularly limited.

In preparing the composition for preparing the hydrogel, water of 2 to 100 times the total weight of carboxymethyl-chitosan (CM-CS) and the hydrophilic synthetic polymer may be mixed. Water of, for example, 5 to 20 times the total weight of carboxymethyl-chitosan (CM-CS) and the hydrophilic synthetic polymer, and preferably, 5 to 15 times, for example, 10 times may be mixed.

Meanwhile, the electron beam may be irradiated at a total dose of 5 to 45 kGy as described above in the hydrogel, for example, at a total dose of 10 to 45 kGy, and preferably, 15 to 45 kGy.

Furthermore, the method for preparing a hydrogel according to the present disclosure may further include drying the crosslinked hydrogel obtained after the crosslinking. In this case, the drying method is not particularly limited, and the drying may be performed out at, for example, 40 to 50° C. for 2 to 4 hours. The drying method is not particularly limited, but may be, for example, natural drying, oven drying, hot air drying, freeze drying, etc., and is preferably performed by freeze drying.

The dried hydrogel may be obtained by additionally performing the drying. A drug-loaded hydrogel for drug delivery may be prepared by further including the loading of the drug into the dried hydrogel. In this case, the drug that may be applied is as described above in the hydrogel.

According to the present disclosure, it is possible to provide a hydrogel with excellent drug delivery ability, biocompatibility, and its own anticancer efficacy, and the hydrogel of the present disclosure meets United States Pharmacopeia (USP) standards for drug release, and thus, may be usefully applied not only to compound drugs that are relatively stable in vivo, but also may be usefully applied even when bio-drugs with physiological activity, such as peptides and proteins, need to maintain stability in vivo.

Hereinafter, the present disclosure will be described in more detail through specific examples. The following examples are merely illustrative to help the understanding of the present disclosure, and the scope of the present disclosure is not limited thereto.

### Example

1. Preparation of Hydrogel

(1) Material

In order to prepare the hydrogel of the present disclosure, the following materials were prepared.

CM-CS (90% deacetylated) was purchased from ChemCruz Biochemicals, Dallas, Tex., USA. PVP (Luviskol-K90, Mw 780,000-1,320,000 Da) was purchased from BASF GmbH, Germany. Sodium chloride (NaCl), potassium chloride, calcium chloride (CaCl₂)), sodium hydroxide, sodium acetate, and monopotassium phosphate (KH₂PO₄) were purchased from Sigma Aldrich (Yongin, Seoul). Ethanol, acetic acid 99.7), boric acid, and hydrochloric acid from Daejung Chemical & Metals Co. Ltd., Siheung, South Korea were used.

(2) Preparation of Hydrogel

CM-CS (3 g) and PVP (6 g) were added to 90 mL of distilled water and homogenized by high-speed disperser (PRIMIX, T.K. HOMOMIXER MARK II, model 2.5) at 3000 rpm. Thereafter, the solution was stored in a water bath (Wisd. DAIHAN Scientific) at 45° C. for 1 hour. The crosslinking between the CM-CS and the PVP was performed by an electron beam accelerator (ELV8-electron accelerator, energy of 2.5 MeV, beam power of 100 kW, beam current of 10 mA, cart speed of 20 m/min) at the Advanced Radiation Research Institute (Jeongeup, Korea). The irradiation of the electron beam was performed at a distance of 40 cm between the sample and the beam source at doses of 15 kGy, 30 kGy, and 45 kGy in air. Thereafter, the crosslinked hydrogel was cut into small squares (5×5 mm) and lyophilized under vacuum (VirTis freeze dryer model: freezemobile 25 EL) at a pressure <20 mT for 72 hours and a temperature of −80° C. The hydrogels prepared at each of the above doses are interchangeably referred to as Example 1 (15 kGy), Example 2 (30 kGy), and Example 3 (45 kGy).

2. Experiment on Physical Properties of Hydrogel

All analyses below were each performed three times, and the obtained data were expressed as mean±standard deviation (SD) using Origin Pro version 9.1 software. Statistical significance was compared using student t-test. Each value (p<0.05) was drawn as statistically significant.

(1) Fourier Transform Infrared Spectroscopy (FTIR) Analysis

FTIR spectra of the CM-CS, the PVP, and the crosslinked hydrogel were evaluated with an ATR-FTIR (Bruker VERTEX 70, Bruker Axs. Inc., Karlsruhe, Germany) spectrometer, and measured in a wavenumber range of 550-4000 cm⁻¹, and at a resolution of 4 cm⁻¹ and a scan rate of 128 scans per sample. For the preparation of the sample, the hydrogel was crushed and pellets were prepared using a compression set.

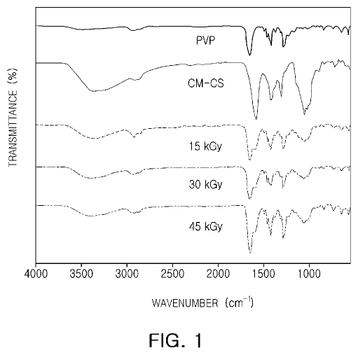

The spectra of the CM-CS, the PVP, and the crosslinked hydrogel according to the present disclosure are as illustrated in FIG. 1. In FIG. 1, the PVP shows characteristic peaks of C—H bending (planar outer ring) at 962 cm⁻¹, C—N stretching at 1286 cm⁻¹, CH₂ including C—H bending at 1461.31 cm⁻¹, a pyridine group including C═O stretching vibration at 1654 cm⁻¹, stretching vibration of C═O at 1654 cm⁻¹, C—H stretching at 2594 cm⁻¹, and a broad band around 3500 cm⁻¹ caused by O—H stretch due to the hydrophilic nature of the PVP.

In FIG. 1, in the case of the carboxymethyl-chitosan (CM-CS), a moderate band at 1423 cm⁻¹ corresponds to a symmetrical transformation of carboxylate ion (COO—), whereas a strong band at 1600 cm⁻¹ corresponds to an asymmetric axial strain (COO—) of the carboxylate ion. In addition, a broader characteristic band is exhibited at about 3440 cm⁻¹ due to the hydrophilic nature induced by the carboxymethyl group.

The crosslinking between the CM-CS and the PVP was confirmed by analyzing the spectrum of the crosslinked hydrogel before the crosslinking. In the case of the crosslinked hydrogel in FIG. 1, the characteristic peaks of the pyridine group including the C—H bending (planar outer ring) at 962 cm⁻¹, the C—N stretching at 1286 cm⁻¹, and the C═O stretching vibration at 1654 cm⁻¹ may confirm the presence of the crosslinking in the hydrogel.

(2) Swelling Test

1) Swelling Test in Distilled Water

Swelling analysis of the prepared hydrogel was performed in distilled water. A pre-weighed sample (45 mg) was stored in a vail and 50 mL of distilled water was added for the sample to be submerged in 50 ml of distilled water. Thereafter, the distilled water was removed every 10 minutes, and the vail was completely dried with tissue paper. Then, the swollen sample was then precisely weighed using a calibrated analytical balance. The swelling exponent was calculated by the following Equation (2).

Swelling exponent of each sample (g/g)=(Wₛ−W_{d})/(W_{d}) Equation (2)

Here, Wₛ represents the swollen hydrogel, and W_{d} represents the dried hydrogel.

Swelling immediately before the weight of the swollen hydrogel starts to decrease is the maximum swelling limit of the prepared hydrogel.

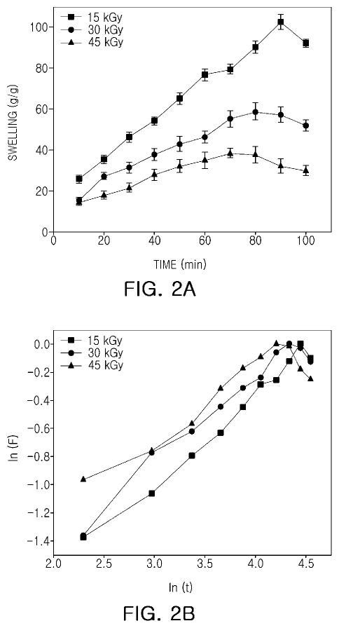

As a result, as can be seen in FIG. 2A, the expansion reaction exhibited time dependence, and crosslinking occurred between the polymers due to irradiation and a porous structure was formed to be responsible for moisture absorption. Meanwhile, it was found that the swelling properties decreased as the radiation dose increased. As such, the degree of crosslinking and the swelling exponent have an inverse relationship. That is, the swelling exponent is affected by the degree of crosslinking.

Meanwhile, the swelling of the hydrogel follows a water diffusion mechanism, and may be calculated by the following Equation (3).

F=ktⁿ Equation (3)

Here, n denotes a swelling exponent, k denotes a swelling rate constant, and F denotes partial swelling determined by a ratio of Wt (swelling at instant t) and W_{eq} (equilibrium time of swelling). These n and k values were extracted from the hydrogel swelling data in the distilled water.

A graph related to the relationship between ln(F) and In(t) was illustrated in FIG. 2B, and diffusion parameter values were summarized in the table below.

TABLE 1

| | Example | Example | Example |
|---|---|---|---|
| Parameter | 1(15 kGy) | 2(30 kGy) | 3(45 kGy) |
| n | 0.613 | 0.569 | 0.408 |
| Y intercept | −2.84857 | −2.56635 | −1.88892 |
| K | 0.057 | 0.076 | 0.151 |
| Standard error in | 0.02669 | 0.04153 | 0.06265 |
| slope | | | |
| Regression, % | 0.99251 | 0.97936 | 0.91728 |
| Adjacent R square | 0.9832 | 0.95405 | 0.82159 |
| Gel fraction, % | 78 ± 3.24 | 83 ± 2.57 | 87 ± 3.11 |
| Porosity, % | 43.78 ± 0.57 | 47.28 ± 0.35 | 59.81 ± 0.74 |

In this case, n is an important factor to evaluate the release phenomenon, and when the swelling exponent n is 1, n denotes the release mechanism of Case II, and when n>0.5, the pattern is non-Fickian, and when n≤0.5, the pattern is related to the Fickian.

As shown in the above Table 1, it was confirmed that the hydrogel of Example 1 exhibited an n value greater than that of the hydrogels of Examples 2 and 3, which indicates that the hydrogel of Example 1 has a higher water absorption capacity.

On the other hand, it was confirmed that the gel content increased as the degree of crosslinking increased.

On the other hand, the crosslinked hydrogel exhibited an increase in porosity (%) with increasing radiation dose, and this increase in porosity is due to the presence of hydrogen bonds and interconnectivity of the hydrogel to stabilize the porous structure and generate porous channels.

2) Swelling Test in pH Solution

Using acetic acid, boric acid, hydrochloric acid, potassium chloride, monopotassium phosphate, potassium hydroxide, sodium acetate, and sodium hydroxide in the required proportions, different pH 2, 4, 6, 8 and 10 buffers were prepared, and a pH meter (METTLER TOLEDO, Seven2Go) was used to monitor. The swelling exponent (g/g) of the pH solution was performed until equilibrium was reached for each sample. The above process was performed three times for each sample to determine the standard error.

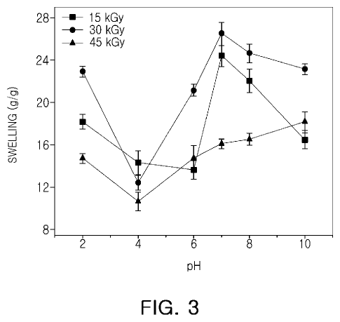

The results were illustrated in FIG. 3, and as illustrated in FIG. 3, in the hydrogel of the present disclosure, the swelling decreased as the pH increased, whereas the neutral pH response was the maximum. The swelling decreased again when the neutral pH was reached. Among them, Example 2 (30 kGy) exhibited the maximum swelling (26.51 g/g) in a neutral environment (pH=7).

3) Swelling Test in Salt Solution

As the swelling may vary depending on the concentration of the salt, in salt solutions including monovalent salt NaCl and divalent salt CaCl₂) prepared at different molar concentrations of 0.2 M, 0.4M, 0.6M, 0.8M, and 1.0M, the swelling exponent (g/g) was performed until equilibrium was reached for each sample. The above process was performed three times for each sample to determine the standard error.

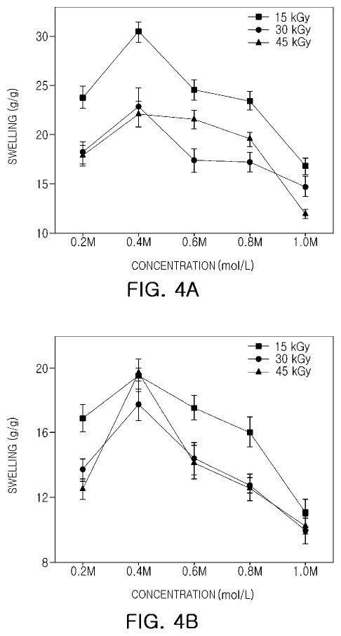

The swelling of the hydrogels of Examples 1 to 3 was measured in NaCl and CaCl₂) aqueous solutions, respectively, and the results were illustrated in FIG. 4A in the case of an aqueous NaCl solution and were shown in FIG. 4B in the case of an aqueous CaCl₂) solution.

As illustrated in FIGS. 4A and 4B, the maximum swelling (30.42 g/g) in the case of NaCl saline solution was observed for the 15 kGy sample at 0.4 M NaCl molar concentration and the maximum swelling (19.62 g/g) in the case of CaCl₂) was observed in 45 kGy samples at 0.4 M NaCl molar concentration. It may be inferred that the swelling value of the NaCl saline solution is greater than that of the CaCl₂) saline solution. This trend is contrary to the general tendency that the CaCl₂) saline solution of the same molar concentration has a swelling value greater than the NaCl saline solutions at the same molar concentration.

As the molar concentration of the salt increased, the swelling behavior of the hydrogel decreased. This is because the charge screening effect occurred as the osmotic pressure between the external solvent and the hydrogel decreased as the concentration of the salt increased. The divalent cations (Ca²⁺) in the CaCl₂ saline solution generate a network structure with a polymer, and the expansion properties increase due to the higher porosity at lower molar concentrations of CaCl₂.

3. Confirmation of In Vitro Cytocompatibility and Anticancer Efficacy of Hydrogel

In vitro cytocompatibility analyses were performed using the ISO 10993-5 method. To prepare an extraction medium, a hydrogel crushed specimen (200 mg) was dispersed in phosphate buffered saline (PBS), stirred in the dark for 24 hours, and then centrifuged at 13,000 rpm for 5 minutes, and the supernatant was filtered with a 0.2 μm syringe filter. The filtered supernatant was mixed with Dulbecco's Modified Eagle's medium (DMEM) to prepare 0.12, 0.25, 0.5, and 1% concentrations.

RAW 264.7 cells and cancerous AGS cells were cultured in DMEM medium containing 10% fetal bovine serum (FBS), 1% penicillin, and streptomycin (PS) at 37° C. and 95% CO₂ and 5% O₂ conditions for 24 hours. Passage growth was continued until the cell seeding density reached 2×10⁵ cells/well. After 24 hours, the culture medium was removed and the extraction solution (diluted twice in the culture medium) was added to a 96-well plate and incubated in incubated oven (under 95% CO₂ and 5% O₂ conditions) at 37° C. for 24 hours. After the incubation, live/dead dyeing (LIVE/DEAD Viability/Cytotoxicity Kit, Molecular Probes Inc., Eugene, Oreg., USA) was performed and images were acquired using a fluorescence microscope (DMI3000B, Leica, Germany). Images were merged using Image J (NIH, MD, USA). Cells were exposed to graded concentration and incubated for 48 hours. Thereafter, the medium was removed from the cells, replaced with a fresh medium containing MTT (0.5 mg/mL), and incubated again at 37° C. for 3 hours. The MTT solution was removed and 100 μL of DMSO was added to dissolve the insoluble formazan crystals formed in the mitochondria of viable cells. The plate was incubated with the DMSO for 5 minutes, and the absorbance was measured in a microplate reader to measure cell viability.

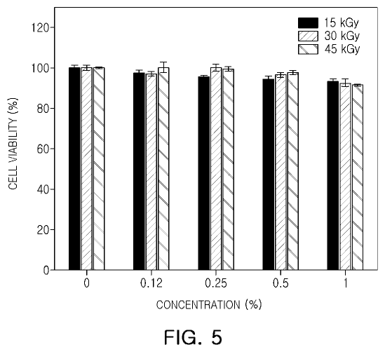

The results of the cell viability (%) for the RAW 264.7 cell line determined by MTT analysis were illustrated in FIG. 5. The observed cell viability (%) of all samples at various concentrations reached >90%, which indicates good cytocompatibility. Among all the samples, the hydrogel of Example 3 to which 45 kGy of radiation was irradiated exhibited excellent cell viability (100.2±2.6%) in the extraction medium at a concentration of 0.12%. These findings support that hydrogels dominate with excellent biocompatibility and noncytotoxicity due to the presence of biocompatible polymers.

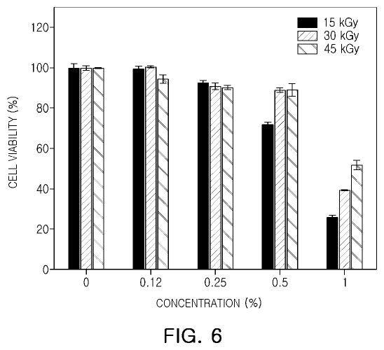

On the other hand, the cytotoxicity to AGS was dose-dependent as illustrated in FIG. 6. As the concentration of the sample increased, the cell viability (%) decreased. For the 15 kGy sample, the cell viability at 0.25, 0.5, and 1% concentrations was 92.6±1.2, 72.2±0.8, and 26.1±0.8%, respectively. In all the samples, the significant decrease in the cell viability (%) could be observed at higher concentrations.

4. Drug Loading and Analysis of Hydrogels

(1) Loading of Drug in Hydrogels

Each lyophilized sample (25 mg) was placed in 100 mg/25 mL PBS of the antibiotic drug kanamycin sulfate for 48 hours. The hydrogel sample exhibited swelling properties and the drugs were injected through the sorption method. Then, it was placed in dark conditions for complete drying. The drug solutions were analyzed using a UV/vis spectrophotometer (Thermo electron corporation model: GEAESYS 10uv scanning) before and after the incubation. The drug loading was calculated according to the following Equation (1).

$$Drug Loading - ( \frac{Amax , d - Amax , t}{Amax , t} ) \times 100 \qquad Equation ( 1 )$$

Amax, d and Amax, t denote the maximum absorbance of the drug solution before and after incubation of the hydrogel sample.

(2) Drug Loading Efficiency and Release Analysis

The drug release analysis of the hydrogel obtained in Example 1 loaded with the drug according to 4.(1) (loaded at a content of 67.23±4.84% by weight) was performed by storing the drug-loaded sample in 100 mL of PBS for 168 hours in a shaking incubator (Vision Sciences, Model VS-8480) at 100 rpm and 37° C. 5 mL aliquots were taken every 12 hours and the PBS volume was replenished by adding an equal amount of fresh solution. The release analysis was performed at 210 nm using a UV/vis spectrophotometer (Thermo electron corporation model: GEAESYS 10uv scanning). A standard drug solution of kanamycin sulfate (100 ppm) in the PBS was used as a reference standard.

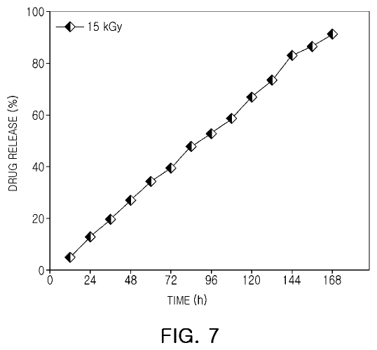

The cumulative drug release analysis of kanamycin in the PBS (pH=7.4) over time was illustrated in FIG. 7. The hydrogel of the present disclosure exhibited the controlled release tendency of the drug over time, and it was confirmed that more than 90% of the drug was released within 168 hours at pH 7.4. The stability of the physical interactions and hydrogen bonds in the network structure of the hydrogel may be protected because the protonation of the —NH₂ group of the CM-CS and the N of the PVP are limited to pH 7.4.

As set forth above, according to an exemplary embodiment in the present disclosure, it is possible to provide a hydrogel with excellent drug delivery ability, biocompatibility, and its own anticancer efficacy, and the hydrogel of the present disclosure meets United States Pharmacopeia (USP) standards for drug release, and thus, may be usefully applied not only to compound drugs that are relatively stable in vivo, but also may be usefully applied even when bio-drugs with physiological activity, such as peptides and proteins, need to maintain stability in vivo.

While exemplary embodiments have been shown and described above, it will be apparent to those skilled in the art that modifications and variations could be made without departing from the scope of the present disclosure as defined by the appended claims.

What is claimed is:

1. A hydrogel including carboxymethyl-chitosan (CM-CS) and a hydrophilic synthetic polymer, and wherein the hydrogel is crosslinked by electron beam irradiation.

2. The hydrogel of claim 1, wherein the hydrophilic synthetic polymer includes at least one selected from the group consisting of polymethacrylic acid, carboxyvinyl polymer, polyacrylamide, polyethylene oxide, polyethyleneimine polyethylene glycol, polyvinyl alcohol, polyacrylate, and polyvinylpyrrolidone.

3. The hydrogel of claim 1, wherein the hydrogel includes the carboxymethyl-chitosan (CM-CS) and the hydrophilic synthetic polymer in a ratio of 1 to 3 parts by weight of the hydrophilic synthetic polymer per 1 part by weight of the carboxymethyl-chitosan.

4. The hydrogel of claim 1, wherein the electron beam is irradiated with a total dose of 5 to 45 kGy.

5. The hydrogel of claim 1, wherein the hydrogel is loaded with 1 to 75 parts by weight of the drug per 100 parts by weight of the hydrogel.

6. The hydrogel of claim 5, wherein the drug includes at least one selected from the group consisting of ampicillin and kanamycin monosulfate monohydrate.

7. A method for preparing a hydrogel, comprising:
preparing a composition for preparing the hydrogel by mixing carboxymethyl-chitosan (CM-CS) and a hydrophilic synthetic polymer with water; and
crosslinking the hydrogel by irradiating the composition for preparing the hydrogel with an electron beam.

8. The method of claim 7, further comprising drying the crosslinked hydrogel obtained after the crosslinking.

9. The method of claim 7, wherein the electron beam is irradiated with a total dose of 5 to 45 kGy.

10. The method of claim 8, further comprising loading a drug to the dried hydrogel.

11. The method of claim 10, wherein the drug includes at least one selected from the group consisting of ampicillin and kanamycin monosulfate monohydrate.
